(19) United States
(12) Patent Application Publication
(10) Pub. No.: US 2014/0025100 A1
(43) Pub. Date: Jan. 23, 2014

(54) RE-SHAPING INTRAGASTRIC IMPLANTS

(71) Applicant: Allergan, Inc., Irvine, CA (US)

(72) Inventors: Mitchell H. Babkes, Santa Clarita, CA (US); Zachary Dominguez, Santa Barbara, CA (US); Christopher Mudd, Ventura, CA (US); Joseph Raven, Tigard, OR (US)

(73) Assignee: Allergan, Inc., Irvine, CA (US)

(21) Appl. No.: 14/034,177

(22) Filed: Sep. 23, 2013

Related U.S. Application Data
(63) Continuation of application No. 13/270,016, filed on Oct. 10, 2011, now abandoned.
Application No. 13/270,016 is a continuation-in-part of application No. 12/902,085, filed on Oct. 11, 2010.
(60) Provisional application No. 61/485,009, filed on May 11, 2011.
(60) Provisional application No. 61/394,592, filed on Oct. 19, 2010.

(51) Int. Cl. A61F 5/00

(52) U.S. Cl. CPC A61F 5/0036
USPC 606/192

(57) ABSTRACT

The present application provides implantable intragastric devices for the treatment of obesity. The intragastric devices advantageously act as a volume-occupying device, and is able to survive implantation in a patient's stomach for a year or longer. In addition, the intragastric devices may be configured to stimulate an inner stomach wall and/or temporarily block the pylorus to slow gastric emptying and/or be rotationally variant, thereby encouraging different stimulation points on the inner wall of the stomach and limiting the stomach's ability to adapt over long term implantation. The intragastric devices may reshape the stomach cavity, such as by pushing on opposite sides so as to “planarize” the stomach. For instance, the device may be an inflated disk, or an implantable loop or a springy coil that may be straightened for delivery/extraction yet assume the loop or coil shape upon implant.

### RELATED APPLICATIONS

The present application is a continuation of U.S. patent application Ser. No. 13/270,016, filed Oct. 10, 2011, which is a continuation-in-part of U.S. patent application Ser. No. 12/902,085, filed Oct. 11, 2010, and also claims priority under 35 U.S.C. §119 to U.S. Provisional Application No. 61/485,009, filed May 11, 2011, and to 61/394,592, filed Oct. 19, 2010, the disclosures of which are incorporated by reference herein.

### FIELD OF THE INVENTION

The present application relates, in general, to devices and methods for controlling obesity, and, more particularly, to an intragastric device designed to promote satiety by occupying volume in a patient's stomach.

### BACKGROUND OF THE INVENTION

Over the last 50 years, obesity has been increasing at an alarming rate and is now recognized by leading government health authorities, such as the Centers for Disease Control (CDC) and National Institutes of Health (NIH), as a disease. In the United States alone, obesity affects more than 60 million individuals and is considered the second leading cause of preventable death. Worldwide, approximately 1.6 billion adults are overweight, and it is estimated that obesity affects at least 400 million adults.

Obesity is caused by a wide range of factors including genetics, metabolic disorders, physical and psychological issues, lifestyle, and poor nutrition. Millions of obese and overweight individuals first turn to diet, fitness and medication to lose weight; however, these efforts alone are often not enough to keep weight at a level that is optimal for good health. Surgery is another increasingly viable alternative for those with a Body Mass Index (BMI) of greater than 40. In fact, the number of bariatric surgeries in the United States is projected to reach approximately 400,000 annually by 2010.

Examples of surgical methods and devices used to treat obesity include the The LAP-BAND® (Allergan, Inc., Irvine, Calif.) gastric band and the LAP-BAND AP® (Allergan, Inc., Irvine, Calif.) gastric band. However, surgery might not be an option for every obese individual; for certain patients, non-surgical therapies or minimal-surgery options are more effective or appropriate.

In the early 1980s, physicians began to experiment with the placement of intragastric balloons to reduce the size of the stomach reservoir, and consequently its capacity for food. Once deployed in the stomach, the balloon helps to trigger a sensation of fullness and a decreased feeling of hunger. These devices are designed to provide therapy for moderately obese individuals who need to shed pounds in preparation for surgery, or as part of a dietary or behavioral modification program. These balloons are typically cylindrical or pear-shaped, generally range in size from 200-500 ml or more, are made of an elastomer such as silicone, polyurethane, or latex, and are filled with air, an inert gas, water, or saline.

One such inflatable intragastric balloon is described in U.S. Pat. No. 5,084,061 and is commercially available as the BioEnterics Intragastric Balloon System (“BIB System”, sold under the trademark ORBERA). The BIB System comprises a silicone elastomer intragastric balloon that is inserted into the stomach and filled with fluid. Conventionally, the balloons are placed in the stomach in an empty or deflated state and thereafter filled (fully or partially) with a suitable fluid. The balloon occupies space in the stomach, thereby leaving less room available for food and creating a feeling of satiety for the patient. Clinical results with these devices show that for many obese patients, the intragastric balloons significantly help to control appetite and accomplish weight loss.

Placement of such balloons is temporary, and such balloons are typically removed after about six months. One means of removing the balloon is to deflate it by puncturing the balloon, and either aspirating the contents of the balloon or allowing the fluid to pass into the patient's stomach. Alternatively, if the balloon is left in place beyond its designed lifetime, the acids present in a patient's stomach may erode the balloon to the point where it self-deflates. When this occurs, the deflated balloon may pass naturally through the patient's digestive system and be expelled through the bowel. For instance, McGhan, U.S. Pat. No. 6,733,512, describes a self-deflating intragastric balloon that includes a biodegradable inflation valve. After a certain residence time in the stomach, the valve starts to leak and eventually the balloon deflates and passes though the patient's digestive tract.

Despite the advances in the design of intragastric balloons, there remains a need for improved medical systems, apparatus and uses thereof for treating obesity and/or obesity-related diseases, and more specifically, to devices designed to stimulate the internal surfaces of the stomach to induce a feeling of satiety.

### SUMMARY OF THE INVENTION

The medical systems, apparatus and uses thereof for treating obesity and/or obesity-related diseases described herein relate to intragastric implant devices designed to stimulate internal surfaces of the stomach. This pressure or stimulation generally promotes a feeling of satiety reducing the amount of food consumed or digested by the patient. The medical systems, apparatus and uses thereof for treating obesity and/or obesity-related diseases described herein may also relate to reducing the space in the stomach, thus advantageously reducing the amount of food consumed or digested by the patient. The intragastric devices described herein may be implanted transorally, through the esophagus and into the patient's stomach without surgery or using only a minimally invasive surgical procedure. At the conclusion of treatment, the intragastric device may be retrieved gastroendoscopically. The intragastric device improves the overall efficacy of transoral obesity reducing devices by achieving a substantial reduction in device weight and may include an identical or improved space-occupying volume when compared to existing devices (e.g., the Orbera® System).

In a first embodiment, an intragastric obesity treatment implant that stimulates the stomach walls comprises an inflatable member having an inflated size that will not pass through the pyloric sphincter and a volume within the stomach of at least 400 ml. The implant is made of a material that will resist degradation over a period of at least six months within the stomach and is formed as a substantially planar disc having opposed faces and a peripheral surface and a maximum width larger than the contracted width of an adult stomach so alter the shape of the stomach to conform somewhat to the inflatable member. The implant is formed of a material which permits it to be compressed when deflated into a substantially linear delivery configuration.

The implant may further include a plurality of dimples on each opposed face of the substantially planar disc defining points at which the opposed faces are connected through an inner cavity to help retain the substantially planar disc shape. In one embodiment, the opposed faces and peripheral surface are smooth, wherein in another version at least some of the opposed faces and peripheral surface are irregular in a pattern selected from the group consisting of a rounded protrusion, a quill-like extension, a recess, and combinations thereof. The opposed faces may be generally circular with a diameter, and the peripheral surface defines a thickness that is less than one-half the diameter. In one embodiment, the implant is constructed out of a material selected from a group consisting of a rubber, a fluorosilicone, a fluoroelastomer, a thermoplastic elastomer, and combinations thereof.

Another intragastric obesity treatment implant that stimulates the stomach walls disclosed herein includes an elongated member having a relaxed configuration that forms a coil. Opposite free ends of the coil overlap one another to permit constriction of the coil. The coil has a diameter that generally fits within the stomach of an adult patient so as to contact the interior stomach walls upon contraction thereof, and the elongated member is formed of a material which permits it to be stretched into a substantially linear delivery configuration and that will resist degradation over a period of at least six months within the stomach. The coil in its relaxed configuration preferably has a diameter of between about 15-16 cm. The coil may comprise an inner pre-formed wire such as Nitinol placed inside a soft and fairly flexible plastic tubular sheath. The two free ends of the wire are desirably captured by two end caps that close open ends of the sheath, and the assembled components of the wire, sheath and end caps are preferably coated with a flexible compound that is resistant to stomach juices. Alternatively, the wire is embedded within the sheath with no hollow spaces therebetween.

In a still further intragastric obesity treatment implant disclosed herein, an elongated hollow member has a relaxed configuration that forms a loop, opposite free ends of the loop being adapted to connect together, and the loop having a diameter that generally fits within the stomach of an adult patient so as to contact the interior stomach walls upon contraction thereof. The elongated hollow member is formed of a material which permits it to be stretched into a substantially linear delivery configuration and that will resist degradation over a period of at least six months within the stomach. The elongated hollow member further may include perforations along its length to permit ingress of stomach fluids.

In one embodiment, the elongated hollow member has a distal end connector with a lumen, and a proximal end connector with a lumen and a side aperture spaced from the proximal end connector, the implant further including a tether that extends through the hollow lumen on the distal end connector and is secured therein, and passes in through the proximal end connector lumen and outward through the side aperture, the proximal and distal end connectors being brought together upon pulling the tether taut. The loop may have a diameter of between about 15-16 cm, and further may include a spring placed inside the elongated hollow member to provide resiliency and prevent kinking of the elongated hollow member. The loop may also be constructed by cutting a plastic tube in two to form two smaller diameter half tubes that fit inside a larger diameter whole tube.

In one embodiment, an intragastric device may have a flat profile and configured to planarize the stomach by forming a wall-like divider in the stomach.

In another embodiment, an intragastric device may have a plurality of round protrusions or “bumps.”

In another embodiment, an intragastric device may have a plurality of legs, each of the plurality of legs having an enlarged end portion.

In another embodiment, an intragastric device may be substantially spherical and may include a plurality of protrusions spaced out on an outer surface of the intragastric device.

In another embodiment, an intragastric device may be substantially spherical and may include a plurality of spine-like or quill-like protrusions spaced out on an outer surface of the intragastric device.

In another embodiment, an intragastric device may be substantially spherical and may include a plurality of dimple-like recesses spaced out on an outer surface of the intragastric device.

For each of the embodiments described above, the intragastric device may be further configured to stimulate an inner stomach wall and/or temporarily block the pylorus to slow gastric emptying and/or be rotationally variant to encourage different stimulation points on the inner wall of the stomach, thereby limiting the ability of the stomach to adapt over long term implantation.

### BRIEF DESCRIPTION OF THE DRAWINGS

FIG. 1A is a perspective view of an exemplary intragastric device that is formed with a flattened geometry so as to planarize the stomach.

FIG. 1B is a top view of the intragastric device of FIG. 1A.

FIG. 1C is a side view along a first axis of the intragastric device of FIG. 1A.

FIG. 1D is a side view along a second axis of the intragastric device of FIG. 1A.

FIG. 2 is a perspective view of an alternative intragastric device similar to the flattened device of FIG. 1A but having the two flat faces connected for shape retention, and

FIG. 3 illustrates the flattened device in place within the stomach.

FIG. 4 is a perspective view of a further intragastric obesity treatment device in a straightened configuration for delivery to the stomach, while FIG. 4A is a longitudinal sectional view thereof

FIG. 5 is a perspective view of the device of FIG. 4 wherein opposite ends have been attached to form a loop capable of altering the shape of the stomach cavity from the inside, and FIG. 5A is a longitudinal sectional view thereof.

FIG. 6 is a perspective view of a further intragastric obesity treatment device in place within the stomach in the shape of a loop capable of altering the shape of the stomach cavity, while FIGS. 7A and 7B are plan and perspective views thereof.

FIG. 8 is a perspective view of an intragastric device in accordance with one or more embodiments described herein.

FIG. 9 is a perspective view of an intragastric device in accordance with one or more embodiments described herein.

FIG. 10 is a perspective view of an intragastric device in accordance with one or more embodiments described herein.

FIG. 11 is a perspective view of an intragastric device in accordance with one or more embodiments described herein.

FIG. 12A is a perspective view of an intragastric device in accordance with one or more embodiments described herein.

FIG. 12B is a close up view of the intragastric device of FIG. 12A in accordance with one or more embodiments described herein.

### DETAILED DESCRIPTION

Persons skilled in the art will readily appreciate that various aspects of the disclosure may be realized by any number of methods and devices configured to perform the intended functions. Stated differently, other methods and devices may be incorporated herein to perform the intended functions. It should also be noted that the drawing figures referred to herein are not all drawn to scale, but may be exaggerated to illustrate various aspects of the invention, and in that regard, the drawing figures should not be construed as limiting. Finally, although the present disclosure may be described in connection with various medical principles and beliefs, the present disclosure should not be bound by theory.

By way of example, the present disclosure will reference certain implantable obesity treatment devices. Nevertheless, persons skilled in the art will readily appreciate that various aspects of the present disclosure advantageously may be applied to one of the numerous varieties of implantable obesity treatment devices.

In one embodiment, these implantable obesity treatment devices described herein are intended to be placed inside the patient, without invasive surgery, without associated patient risks of invasive surgery and without substantial patient discomfort. Recovery time may be minimal as extensive tissue healing is generally not required. The life span of these obesity treatment devices may be material-dependent upon long-term survivability within an acidic stomach, but is intended to last one year or longer in various embodiments. Moreover, each device described herein is designed to stimulate internal surfaces of the stomach. This pressure or stimulation generally promotes a feeling of satiety reducing the amount of food consumed or digested by the patient. The medical systems, apparatus and uses thereof for treating obesity and/or obesity-related diseases described herein may also relate to reducing the space in the stomach, thus advantageously reducing the amount of food consumed or digested by the patient.

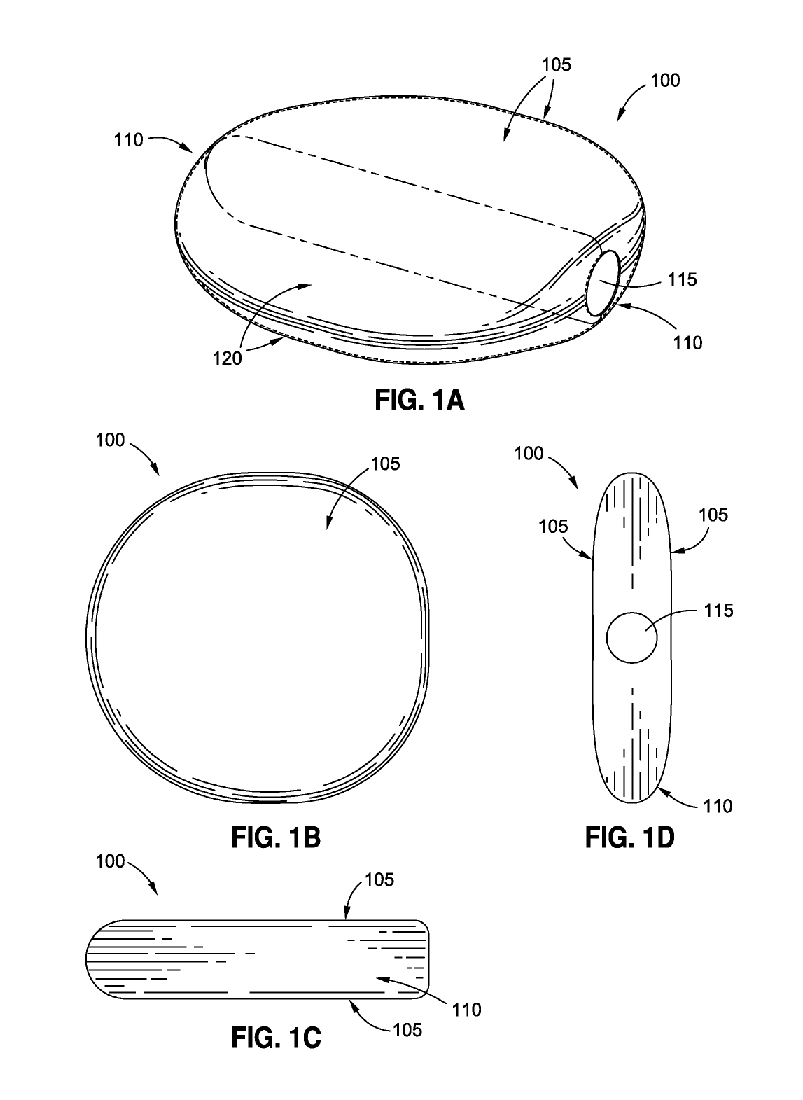

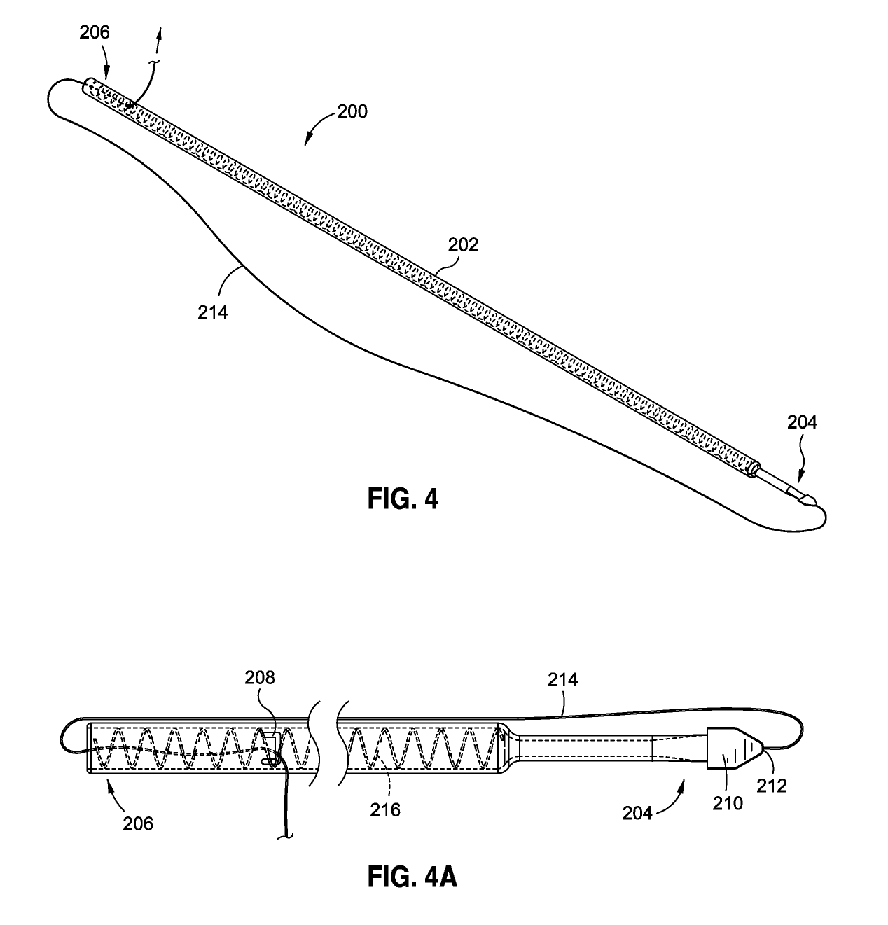

In addition to stimulating the stomach nerves, the devices described herein desirably include geometries that reshape the stomach cavity. Non-uniformity in the cross-sectional shape of the devices can be used to stretch the stomach to a flatter geometry, which in turn reduces the volume capacity of the stomach. For instance, FIGS. 1-3 show rounded, flattened intragastric devices that retain their shape in the stomach. FIGS. 4-7 illustrate tubular members that can be formed into loops for planarizing the stomach. Additionally, a number of the devices described herein can be made rotationally variant, such that movement within the stomach results in certain arbitrary rotational adjustments which causes the device to occupy a different 3-dimensional space and orientation. This encourages different stimulation points as the device moves in the stomach, limiting the ability of the stomach to adapt over long term implantation. Still further, outer bumps, protrusions, quill-like extensions, or other surface irregularities may be provided on any of the various shapes to enhance stimulation of the inner wall of the stomach.

In a first embodiment seen in FIG. 1A, an intragastric device 100 has a flat profile configured to planarize the stomach by forming a wall-like divider in the stomach. More particularly, the geometry of the intragastric device 100 stretches the stomach to a flatter geometry, which causes the volume capacity of the stomach to be substantially reduced. The intragastric device 100 of FIG. 1A includes top and bottom surfaces 105, and a peripheral surface 110 substantially extending around the circumference of intragastric device 100. The peripheral surface 110 may be oval-shaped and the opposed top and bottom surfaces 105 are generally flat. The long dimension of the oval-shaped peripheral surface 110 will be slightly larger than the longest dimension across any opposite walls of the stomach cavity so that the device changes the shape of the stomach cavity. Desirably, the intragastric device 100 can be either inflated, such as a typical intragastric balloon, or can be molded from a thick-walled polymer so as to be able to retain its shape in the stomach. In the latter configuration, through holes may be provided to allow passage of stomach juices.

As shown, in FIG. 1A, the top and bottom surfaces 105 may comprise a plurality of joined panels 120 attached to each other and also to a circular panel 115 on an end substantially perpendicular to the top and bottom surfaces 105. As shown, the intragastric device 100 may be constructed of rubber, fluorosilicones, fluoroelastomers, thermoplastic elastomers or any combinations thereof. In one embodiment, the intragastric device 100 is filled with air/inert gas or a liquid such as silicon. The material(s) utilized allow for a thinner wall thickness and have a lower coefficient of friction. Thinner walls and the lower coefficient of friction allows improved natural passage of the intragastric device 100 through the esophagus during delivery, and also through the gastrointestinal tract should the intragastric device 100 deflate for any reason inside the patient's stomach.

FIG. 1B illustrates a top view of the intragastric device 100 of FIG. 1A. As illustrated, the intragastric device 100 is substantially circular or slightly oval. However, a different geometry may be implemented. For example, an intragastric device incorporating a more pronounced oval or ellipse-shaped top or bottom surface is possible. Desirably, the end having the circular panel 115 is flat, as shown. The circular panel 115 may provide a self-sealing inflation patch, or may represent a fill valve. While not shown, the top and bottom surfaces 105 may further include stimulation features such as rounded bumps or protrusions, quill-like extensions, dimples or recesses, and the like, as described below. These features, upon contact with the inner stomach wall of the patient may trigger hormone release or otherwise aid the patient in feeling full.

FIG. 1C illustrates a side view of the intragastric device 100 of FIG. 1A along one axis of the intragastric device 100. As shown, the thickness of the intragastric device 100 (or width of the peripheral surface 110) is even. However, alternative configurations, including varying thicknesses of the intragastric device 100, may be possible. In one embodiment, the intragastric device 100 has a diameter of about 5-15 cm (2-6 inches) and a thickness which is one-half or less than the diameter, more preferably less than one-third the diameter. In one example, the thickness is between about 1.2-8 cm (0.5-3 inches), with the lower bound matching the lower bound of the diameter, and vice versa. In a preferred embodiment, the intragastric device 100 has a diameter of about 10-15 cm (4-6 inches) and a thickness of between about 2-4 cm (0.75-1.6 inches), with the lower bound matching the lower bound of the diameter, and vice versa.

FIG. 1D illustrates a side view of the intragastric device 100 of Figure lA along an axis of the intragastric device 100. Here, the circular panel 115 is shown in the center of the flat end and does not extend to the end surface 105. In one embodiment, the circular panel 115 may include a port (not shown) for the inflation or deflation of the intragastric device 100.

The size of the intragastric device 100 may be configured such that the entire intragastric device 100 may be insertable transorally through the esophagus and into the stomach without invasive surgery. In one embodiment, the intragastric device 100 may be inserted into the patient's stomach using a standard grabber. Alternatively, the deflated intragastric device 100 may be passed through an access tube placed down the esophagus, which may be lubricated for ease of passage.

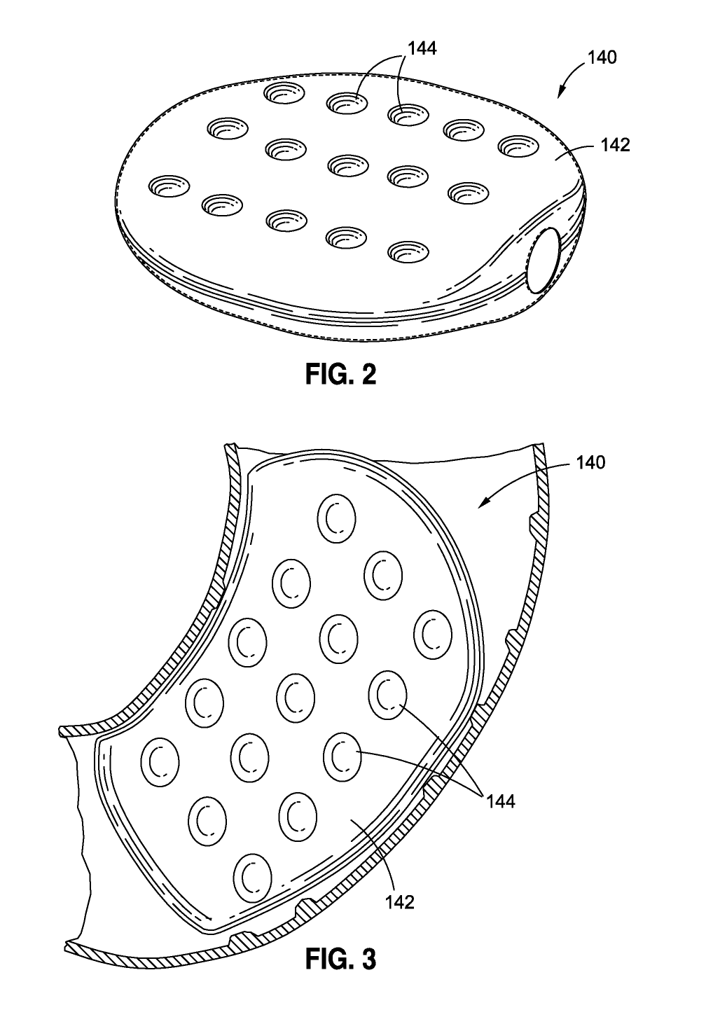

FIG. 2 shows an alternative intragastric device 140 similar to the flattened oval-shaped device 100 of FIG. 1A but having two flat faces 142 connected for shape retention. There are a number of ways to do this; the illustrated embodiment shows a plurality of dimples 144 representing points at which the faces 142 are bonded together, such as by thermal welding. The result resembles upholstered buttons on a couch cushion. The plurality of dimples define points at which the opposed faces 142 are connected to each other through an inner cavity. FIG. 3 shows the device 140 implanted within the stomach. Because of the points of connection between the two flat faces 142, the device 140 better maintains its flattened configuration when inflated and thus better reshapes the stomach cavity. Although not shown, exterior grooves or internal flow passages may be provided through the device to permit passage of food through the stomach. Further, the external surfaces may further include stimulation features such as rounded bumps or protrusions, quill-like extensions, dimples or recesses, and the like, as described below. Upon contact with the inner stomach wall of the patient these irregularities may trigger hormone release or otherwise aid the patient in feeling full.

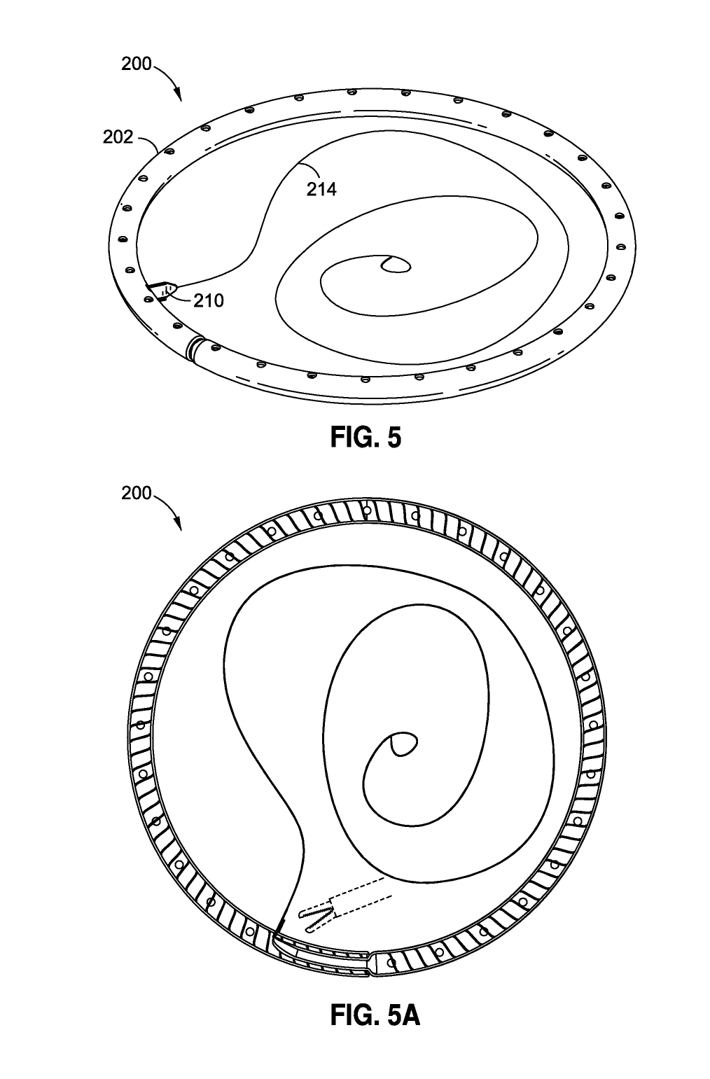

Another alternative satiety-inducing device 200 is shown in FIGS. 4-5, and comprises a solid member in the form of a ring that “planarizes” or flattens the stomach. Satiety is achieved by a two-fold effect: reducing the stomach volume and contacting gastric nerves. The amount of food ingested is restricted, as this device “planarizes” the stomach which decreases the stomach's effective volume and capacity. Since there is less room for food, appetite is suppressed earlier than normal, thus reducing calorie consumption. In addition to reducing consumption potential, early feelings of satiety are also created from the “spring-like” memory retention of the loop that exerts a pressure on the stomach walls. It is believed that gastric nerves respond to pressure applied against the stomach walls in various positions, thus signaling the brain to release hormones that send a signal of satiety.

Referring to FIG. 4, the satiety-inducing loop 200 is shown in a straightened configuration for transoral introduction into the body. The device 200 includes an elongated tubular body 202 having a head end 204, a tail end opening 206, and two opposed slotted body openings 208 adjacent the tail end. The head end 204 defines a flattened barbed head 210 and an opening 212 in a tip thereof. As shown, one end of a tether 214 fastens inside the head end 204, while the majority of the tether extends out of the opening 212 and traverses the outside of the body 202, passing into the end opening 206 and then exiting the body opening 208. A spring 216 placed inside the formed tube provides the formed loop 200 with resiliency and prevents kinking A second embodiment of this invention has no spring inside the tube, otherwise it is configured identically. This configuration is dependent upon arriving at a material having properties that are both stable in the acidic stomach environment for over one year, and also possesses a true spring-like “memory” retention of its own.

The satiety-inducing loop 200 may be easily implanted inside the patient transorally, without invasive surgery (and without the corresponding patient risks inherent in a surgery) and with a minimal recovery time since no extensive tissue healing is required. While in a substantially straight state, as in FIG. 4, the satiety-inducing loop 200 may be inserted through a patient's mouth, down the esophagus and into the stomach while keeping a portion of the string outside the patient's mouth. A standard grabber may be used during the implantation process to assist implantation of loop 200, or the straightened loop 200 may be passed through an access tube placed down the esophagus, which may be lubricated for ease of passage. After the loop 200 is inside the patient's stomach and held in place by the grabber, the physician may pull on the tether 214 to bring the head end 204 to the end opening 206. By continuing to pull on the tether 214, the head end 204 enters end opening 206 and the flaps of arrow-shaped head 210 exits body opening 208, which locks the head end 204 inside the end opening 206, as seen in FIGS. 5 and 5A, thus forming a substantially circular loop. The physician then cuts the tether 214 once the loop 200 is formed, and any remaining tether inside the body may be disintegrated or digested by the juices inside the stomach. Accordingly, the string may be constructed out of a non-toxic substance.

The body 202 of the satiety-inducing loop 200 may be constructed out of polypropylene or other suitable materials for resisting the acidity of the stomach environment. For instance, the satiety-inducing loop 200 may be fabricated by heating either by heating and stretching areas of a plastic tube to be cut in two, so they form two smaller diameter areas that fit inside the large ID, or the smaller areas can be formed by a calibrated differential extrusion process. After sizing and cutting to length, the “arrow”-shaped head end 204 can be heated and stamped into shape with a die. A pre-formed spring 216, if needed for the first embodiment, can then be inserted into the tube. During the arrow-stamping operation, the string/tether 214 must be rolled at one end and inserted into the arrow, then threaded through the opposite end and out a side hole.

In one aspect, the diameter of the loop 200 when in its implanted state is configured to fit the patient's stomach while causing some reshaping thereof. While shown in a substantially circular design, the satiety-inducing loop 200 may be configured to take on any shape, including ovals, quadrilaterals, triangles, and even uncommon or random shapes (where the non-circular shaped loops may include joints that snap into place during the insertion process when the string is pulled). If circular, the diameter of the formed loop 200 is desirably between about 15-16 cm.

The satiety-inducing loop 200 may be configured to be easily removed by a standard grabber. By utilizing the grabber to squeeze the opposing ends of the arrow of the head 210 so that they align with the slot 208, the head 210 may slip back out of the slot, causing it to gently spring back to its original, straight state. Then, using the grabber that is already inserted, the satiety-inducing loop 200 may be grasped and removed back up the esophagus and out the patient's mouth. By adding a radio-opaque additive into the material of the arrow, the head 210 may be seen by an x-ray machine during the removal process.

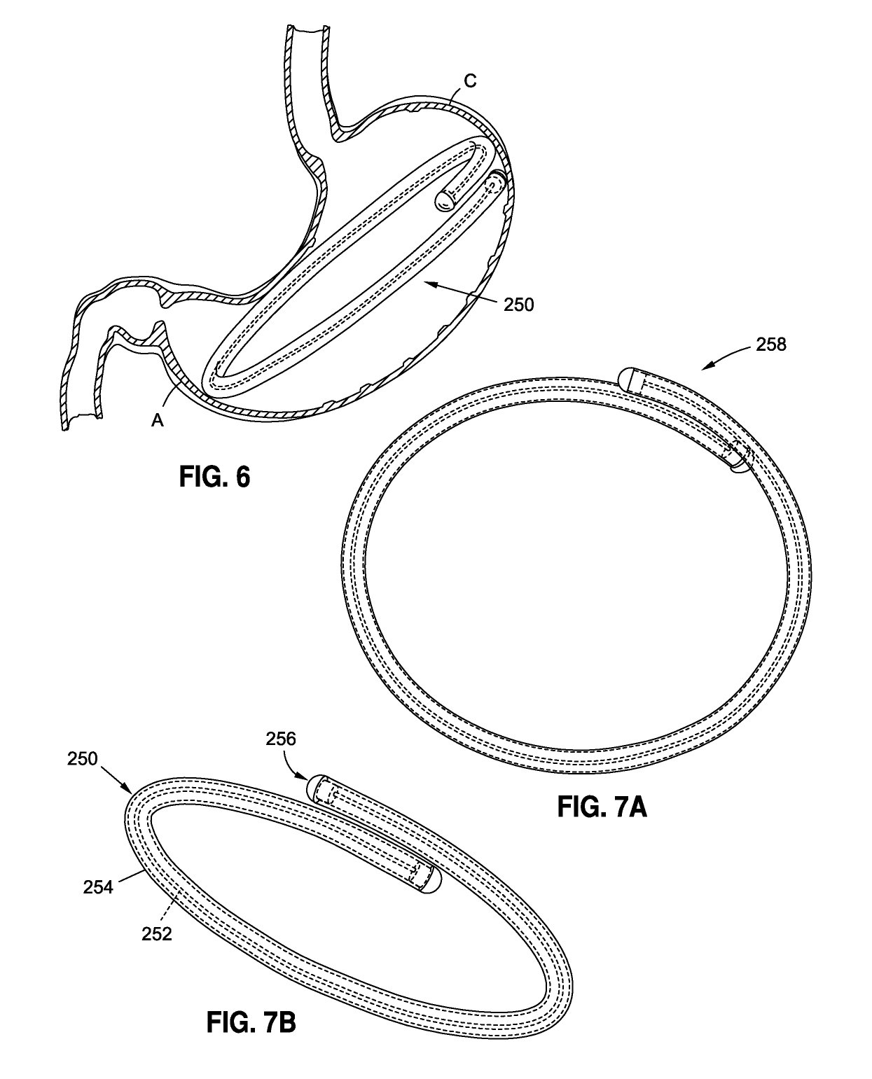

FIG. 6 is a perspective view of a further intragastric obesity treatment device 250 in place within the stomach in the shape of an open resilient coil capable of altering the shape of the stomach cavity. The device 250 generally forms a generally circular coil is shown having a diameter sufficient to extend from approximately the cardia C at the upper end of the stomach to the antrum A at the lower end. For instance, the diameter of the coiled device 250 is desirably between about 15-16 cm.

With reference also to FIGS. 7A-7B, the obesity treatment device 250 preferably comprises an inner pre-formed wire 252 place inside a soft and fairly flexible plastic tubular sheath 254. Two open ends of the sheath 254 are closed with hard plastic, domed end caps 256 that also trap each end of the wire 252. That is, each of the domed end caps 256 preferably includes a small hole that receives one end of the wire 252, which can be secured therein with adhesive or the like. In one embodiment, the diameter of the sheath 254 has a diameter small enough to fit through a delivery tube (not shown) of about 19-20 mm. The domed end caps 256 have a maximum diameter approximately equal to the diameter of the sheath 254, thus providing a smooth junction therebetween. The relatively soft and rounded configuration of the sheath 254 and domed end caps 256 prevents trauma to the stomach walls from the wire 252. The assembled components of the device 250 are preferably coated with a flexible compound that is resistant to stomach juices. This coating, which may be dipped or sprayed on and then post-hardened, is also intended to fully seal the device against fluid ingress. Such a coating will desirably be an elastomeric polymer that will withstand the acidic environment and biological contaminants of the stomach.

In one embodiment, the wire 252 comprises Nitinol shaped in a tight spiral and having partially overlapping ends 258. The wire 52 acts like a flat spring so that when compressed by peristaltic action of the stomach, it is inclined to return to its initial shape, thus applying outward pressure. Squeezing the device 250 on the outside will tend to make the coiled ring momentarily smaller, but compression in the direction along the axis of the coil we have no noticeable effect.

An alternative embodiment of the device 250 comprises a solid, coiled rod, rather than a wire positioned within an outer sheath. To help prevent trauma to the stomach walls, the ends of a solid rod may be capped with bulbs or other such rounded or enlarged members. IN a still further embodiment, a Nitinol wire such as the wire 252 above may be coated with or embedded within a polymer to increase the exterior dimension and provide atraumatic ends. For instance, the device 250 shown above may be constructed this way so that there are no hollow spaces defined within.

As before, the obesity treatment device 250 is intended to be transorally placed, without the need for laparoscopic or other surgical assist, and without any need for piercing or cutting of tissues or physically anchoring the device. The coiled device 250 can freely float, moving as the stomach moves. The outward spring action of the coil 250 is meant to apply pressure to infinitely variable areas of the stomach walls causing all-over stimulation.

Insertion and removal of the obesity treatment device 250 can be done repeatedly. The device 250 relies on the physical property of the internal Nitinol wire to straighten out, then instantly returned to its pre-formed shape. Over-stressing of Nitinol in this configuration is highly unlikely, even when acted upon by repeated, often extreme movement of the stomach. On the other hand, regular stainless steel or other such spring wire might deform when over-stressed by the stomach, or from straightening as needed for insertion/removal.

Additionally, a number of the devices described herein can be made rotationally variant, such that movement within the stomach results in certain arbitrary rotational adjustments which causes the device to occupy a different 3-dimensional space and orientation. This encourages different stimulation points as the device moves in the stomach, limiting the ability of the stomach to adapt over long term implantation.

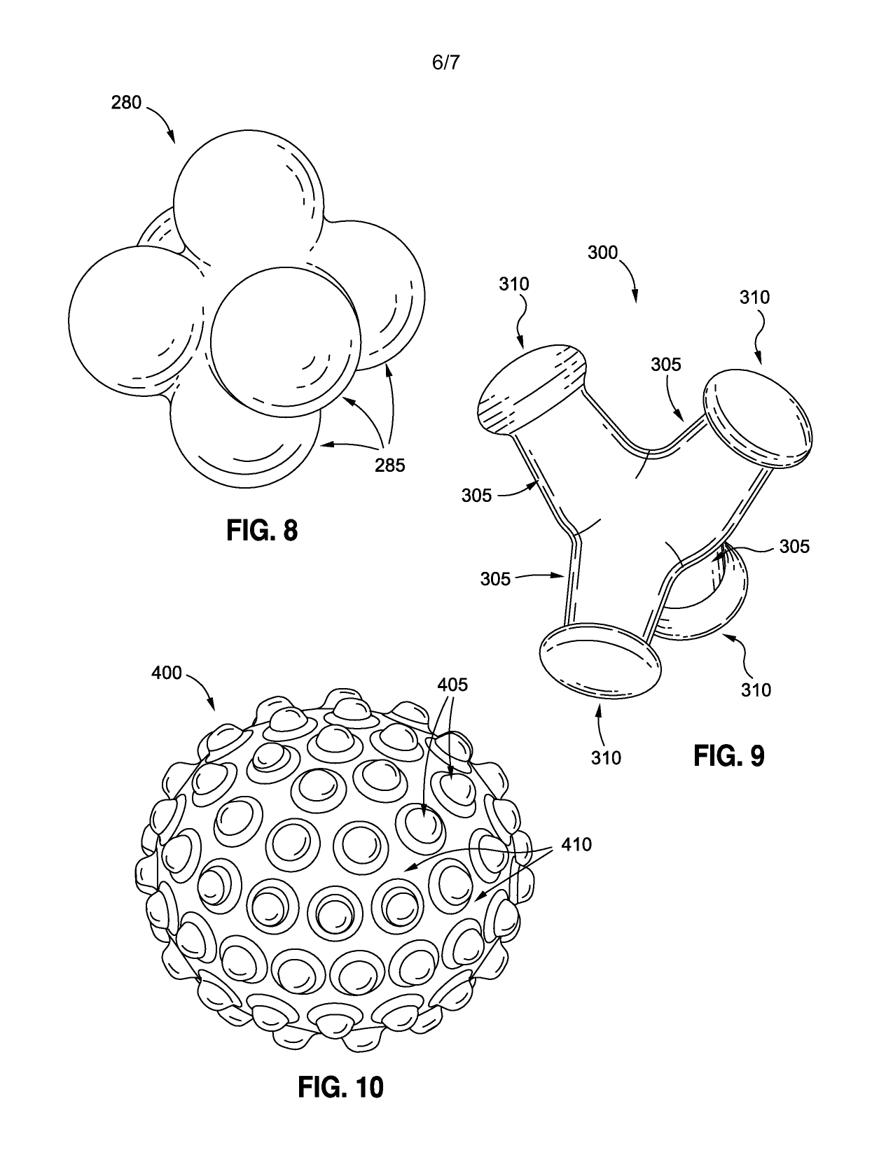

FIG. 8 illustrates another embodiment of an intragastric device 280 of the present application that is rotationally variant. As shown, the intragastric device 280 has a plurality of protrusions 285 or generally spherical “bumps” formed outwards from a center region of the intragastric device 280. While the intragastric device 280 may be sized to fit comfortably inside the patient's stomach, each of the plurality of protrusions 285 may be sized such that it blocks the patient's pylorus temporarily when the protrusion 285 comes into contact with the pylorus, thereby slowing gastric emptying and allowing the patient to feel full for a longer period of time without the protrusion 285 getting stuck or wedged into the pylorus. In addition, the configuration of the intragastric device 280 may produce variations in how the intragastric device 280 sits or rotates inside the patient's stomach. The overall exterior shape of the device is somewhat spherical, encouraging rotation. However, the outwardly projecting spheres that make up the device contact the stomach wall at different locations as the device rotates. Normal stomach contractions thus cause the intragastric device 280 to move around or rotate about the stomach, and due to the device's configuration, different points on the inner stomach walls may be stimulated, thereby limiting the stomach's ability to adapt over a long period of time. The protrusions 285 may be added to a number of the devices described herein.

In one embodiment, the protrusions 285 may be placed in an asymmetrical pattern to further limit the ability of the stomach to adapt over a long period of time.

The intragastric device 280 may be constructed of rubbers, fluorosilicones, fluoroelastomers, thermoplastic elastomers or any combination thereof to improve the durability of the intragastric device 280 inside the patient's stomach. However, the intragastric device 280 may be constructed of a continuous, thin, depressable wall and be hollow inside (filled with air/inert gas) to keep the intragastric device 280 light. Alternatively, the intragastric device 280 may be filled with a liquid gel such as silicon. The material(s) utilized to construct the intragastric device 280 may allow for a thinner wall thickness and have a lower coefficient of friction. Thinner walls and the lower coefficient of friction allow improved natural passage of the intragastric device 280 through the gastrointestinal tract should the intragastric device 280 deflate for any reason inside the patient's stomach.

While not shown, the outer surface of the intragastric device 280 may further include additional stimulation features such as even smaller rounded bumps or protrusions formed on the protrusions 285 (e.g., 10-15 mini-protrusions on each of the six protrusions shown in FIG. 8, equally spread apart and having a substantially similar shape, but with a smaller diameter as compared to the protrusion 285), quill-like extensions, dimples or recesses, and the like. These features, upon contact with the inner stomach wall of the patient may further trigger hormone release or otherwise aid the patient in feeling full.

FIG. 9 illustrates another embodiment of the intragastric device 300. As shown, the intragastric device 300 has four “legs” 305 terminating in rounded or bulbous ends 310. The configuration of the four legs may be asymmetrical as shown. If divided into “the top two legs” and “the bottom two legs”, the “pairs of legs” appear joined at the center and “twisted 90 degrees” to form the configuration as shown. However, this is merely one example of any of a plurality of configurations for any of a plurality of leg numbers.

For example, additional legs may be attached or removed, and/or the configuration may be altered. In addition, each leg portion 305 terminate in the bulbous ends 310. The ends 310, as shown, cap the end of the leg portion 305 and each has a diameter substantially thicker than the diameter of the leg portion 305. Accordingly, the ends 310 may be sized such that it blocks the patient's pylorus temporarily when the ends 310 comes into contact with the pylorus, and thereby slowing gastric emptying and allowing the patient to feel full for a longer period of time without the ends 310 getting stuck or wedged into the pylorus. Again, the device 300 rotates relatively easily within the stomach, especially upon peristaltic motion, and the separated legs 305 and ends 310 therefore contact the stomach wall at different locations on a constantly changing basis. Normal stomach contractions may cause the intragastric device 300 to move around or rotate about the stomach, and due to the device's configuration, different points on the inner stomach walls may be stimulated, thereby limiting the ability of the stomach to adapt over a long period of time. These features can be utilized in a device that looks like the device 300, or can be added to a number of the embodiments described herein, such as the inflated member 100 of FIGS. 1A-1D.

The intragastric device 300 may be constructed of rubbers, fluorosilicones, fluoroelastomers, thermoplastic elastomers or any combination thereof to improve the durability of the intragastric device 300 inside the patient's stomach. However, the intragastric device 300 may be constructed of a continuous, thin, depressable wall and be hollow inside (filled with air/inert gas) to keep the intragastric device 300 light. Alternatively, the intragastric device 300 may be filled with a liquid gel such as silicon. The materials utilized may allow for a thinner wall thickness and have a lower coefficient of friction. Thinner walls and the lower coefficient of friction allows improved natural passage of the intragastric device 300 through the gastrointestinal tract should the intragastric device 300 deflate for any reason inside the patient's stomach.

While not shown, the outer surface of the intragastric device 300 may further include additional stimulation features such as even small rounded bumps or protrusions formed on the ends 310 (e.g., 10-15 mini-protrusions on each of the four ends shown in FIG. 9, equally spread apart and having a substantially similar shape, but with a much smaller diameter as compared to the ends 310), quill-like extensions, dimples or recesses, and the like. These features, upon contact with the inner stomach wall of the patient may further trigger hormone release or otherwise aid the patient in feeling full.

Another option for a number of the intragastric devices disclosed herein is to add exterior stimulation features, such as any raised or depressed geometry which act to stimulate certain portions of the stomach walls. Such features may be particularly effective for those embodiments which stimulate the cardia.

FIG. 10 illustrates another embodiment of the intragastric device 400. As shown, the intragastric device 400 is a substantially spherical object with protrusions or bumps 405 extending outward from the surface of the intragastric device 400. As shown, a plurality of protrusions 405 may be equally spaced apart on the outer surface and interspersed with flat portions 410. In one embodiment, the protrusions 405 do not contact each other. In another embodiment, the protrusions 405 may be of equal heights and diameters. However, the protrusions 405 may be configured to contact each other (and thereby creating space and allowing for more protrusions 405 to be added to the surface). In another embodiment, the protrusions 405 may be configured to have different heights and/or diameters. For example, having protrusions with different heights and/or diameters may be advantageous for preventing the stomach from adjusting to the protrusions 405. The protrusions 405 separately contact the inner walls of the stomach, potentially increasing the stimulation to the surrounding satiety-sensing nerves.

In another embodiment, the size of the intragastric device 400 may be altered. For example, in a uni-intragastric device system, one intragastric device 400 may be implanted into the patient's stomach, and the single intragastric device may be sized accordingly to fit comfortably inside the patient's stomach. However, it is also possible to employ multiple, smaller devices, such as 2 or 3 objects similar to the intragastric device 400. Under this multi-intragastric device system, each intragastric device 400 may be sized such that it blocks the patient's pylorus temporarily when the intragastric device 400 comes into contact with the pylorus, and thereby slowing gastric emptying and allowing the patient to feel full for a longer period of time (and also to prevent intestinal blockage). In addition, the configuration of the intragastric device 400 may produce variations in how the intragastric device 400 sits or rotates inside the patient's stomach. Normal stomach contractions may cause the intragastric device 400 to move around or rotate about the stomach, and due to the device's configuration, different points on the inner stomach walls may be stimulated, thereby limiting the ability of the stomach to adapt over a long period of time.

The intragastric device 400 may be constructed of rubbers, fluorosilicones, fluoroelastomers, thermoplastic elastomers or any combination thereof to improve the durability of the intragastric device 400 inside the patient's stomach. However, the intragastric device 400 may be constructed of a continuous, thin, depressable wall and be hollow inside (filled with air/inert gas) to keep the intragastric device 400 light. Alternatively, the intragastric device 400 may be filled with a liquid gel such as silicon. Regardless, the materials utilized may allow for a thinner wall thickness and have a lower coefficient of friction. Thinner walls and the lower coefficient of friction allows improved natural passage of the intragastric device 400 through the gastrointestinal tract should the intragastric device 400 deflate for any reason inside the patient's stomach.

While not shown, the outer surface of the intragastric device 400 may further include additional stimulation features such as even small rounded bumps or protrusions formed on the protrusions 405 (e.g., 10-15 mini-protrusions on each of the protrusions 405 of FIG. 10, equally spread apart and having a substantially similar shape, but with a much smaller diameter as compared to the protrusions 405), quill-like extensions, dimples or recesses, and the like. These features, upon contact with the inner stomach wall of the patient may further trigger hormone release or otherwise aid the patient in feeling full.

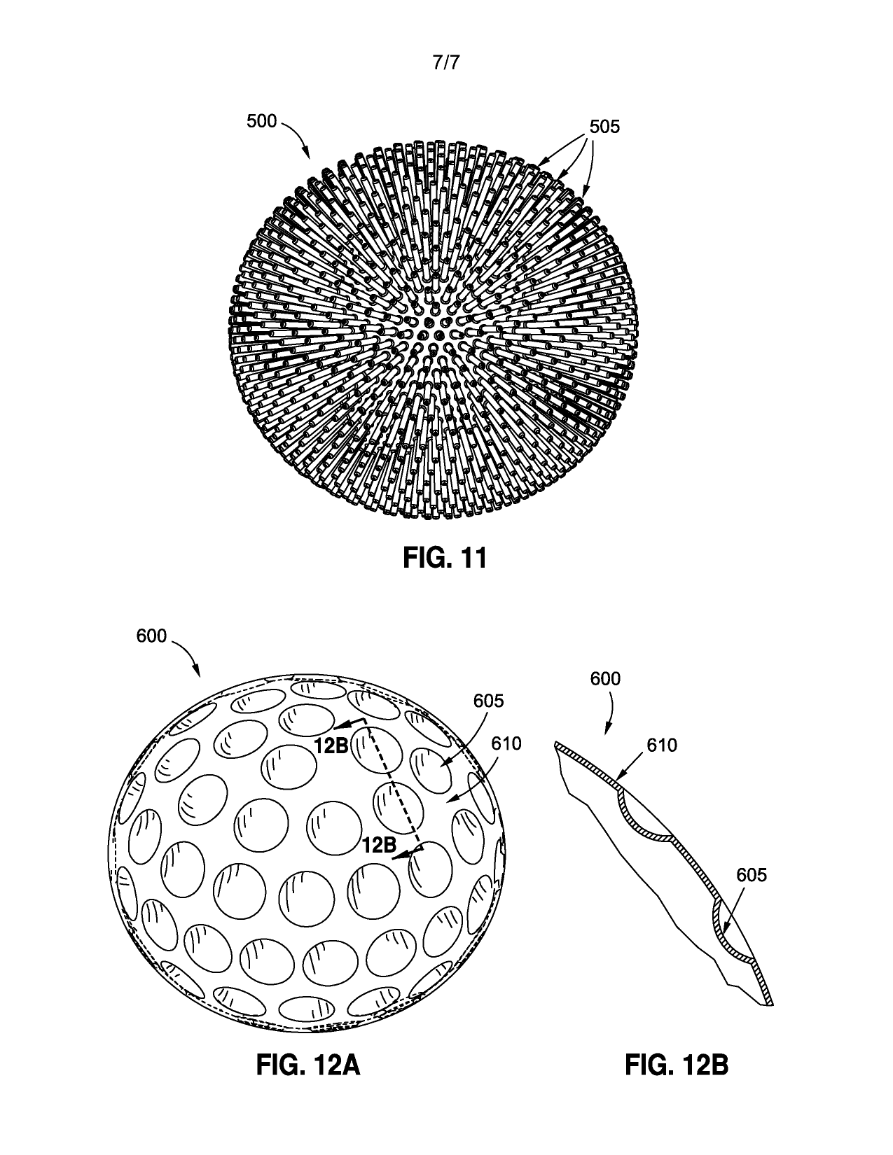

FIG. 11 illustrates another embodiment of the intragastric device 500. As shown, the intragastric device 500 is a substantially spherical object with long flagella or quill-like extensions 505 extending outward from the outer surface of a central region of the intragastric device 500. As shown, a plurality of extensions 505 may be equally spaced apart. In one embodiment, the extensions 505 do not contact each other. In another embodiment, the extensions 505 may be of equal heights and diameters. However, the extensions 505 may be configured to contact each other (and thereby creating space and allowing for more extensions 505 to be added to the surface). In another embodiment, the extensions 505 may be configured to have different heights and/or diameters. For example, having protrusions with different heights and/or diameters may be advantageous for preventing the stomach from adjusting to the extensions 505. In one embodiment, the extensions may be extremely flexible and may bend when a pressure is exerted on the extensions 505 from the inner stomach wall of the patient. Alternatively, the extensions 505 may be stiffer and might not bend as much when a pressure is exerted on the extensions 505 from the inner stomach wall of the patient. In another embodiment, some of the extensions 505 may have a first flexibility while some of the extensions may have a second flexibility. Alternatively, the extensions 505 may be uniformly flexible. In other words, any flexibility of the extensions may be utilized with the intragastric device 500.

In another embodiment, the size of the intragastric device 500 may be altered. For example, in a uni-intragastric device system, one intragastric device 500 may be implanted into the patient's stomach, and the single intragastric device may be sized accordingly to fit comfortably inside the patient's stomach. However, it is also possible to employ multiple, smaller devices, such as 2 or 3 objects similar to the intragastric device 500. Under this multi-intragastric device system, each intragastric device 500 may be sized such that it blocks the patient's pylorus temporarily when the intragastric device 500 comes into contact with the pylorus, and thereby slowing gastric emptying and allowing the patient to feel full for a longer period of time (and also to prevent intestinal blockage). In addition, the configuration of the intragastric device 500 may produce variations in how the intragastric device 500 sits or rotates inside the patient's stomach. Normal stomach contractions may cause the intragastric device 500 to move around or rotate about the stomach, and due to the device's configuration, different points on the inner stomach walls may be stimulated, thereby limiting the ability of the stomach to adapt over a long period of time. In another embodiment of the multi-intragastric device system, different intragastric devices may be implanted into the same patient's stomach at the same time. For example, the intragastric device of FIG. 10 and the intragastric device of FIG. 11 may both be implanted in the patient and may simultaneously work together. One benefit of this approach may be that the stomach will have an even more difficult time adjusting to the intragastric devices 400 and 500 since they are completely different from one another, thereby improving the efficacy of the system.

Referring back to FIG. 11, the intragastric device 500 may be constructed of rubbers, fluorosilicones, fluoroelastomers, thermoplastic elastomers or any combination thereof to improve the durability of the intragastric device 500 inside the patient's stomach. However, the intragastric device 500 may be constructed of a continuous, thin, depressable wall and be hollow inside (filled with air/inert gas) to keep the intragastric device 500 light. Alternatively, the intragastric device 500 may be filled with a liquid gel such as silicon. Regardless, the materials utilized may allow for a thinner wall thickness and have a lower coefficient of friction. Thinner walls and the lower coefficient of friction allows improved natural passage of the intragastric device 500 through the gastrointestinal tract should the intragastric device 500 deflate for any reason inside the patient's stomach.

FIG. 12A illustrates another embodiment of the intragastric device 600. As shown, the intragastric device 600 is a substantially spherical object with recesses or dimples 605 extending inward from the surface of the intragastric device 600. In one embodiment, the intragastric device 600 may be considered to have a surface comprised of recesses 605 and flat portions 610. As shown, a plurality of recesses 605 may be equally spaced apart on the outer surface. As shown, recesses 605 do not contact each other, and may be of equal heights and diameters. In addition to being depressed, the recesses 605 may employ a thinner wall. For example, if the flat portions 610 have a wall thickness of 20 millimeters, the recesses 605 may have a wall thickness of 10 millimeters. With a thinner wall, the recesses 605 may be more susceptible to larger strains.

The intragastric device 600 is effectively triggered in the patient's stomach by stomach contractions. These stomach contractions increase the pressure in the intragastric device 600. FIG. 12B illustrates a close up view of the recesses 605 and the flat portions 610. If the recess 605 is not in contact with the stomach wall or some outside retaining force, the recess 605 with the thinner walls will deform until the recess 605 comes into contact with the stomach wall or comes under the influence of some other outside force. The recess 605 will also stop deforming when no contact is made when the modulus of the material forming the recess is such that the stress in the material is balanced with the internal pressure of the intragastric device 600.

Now, if the recess 605 is in contact with the stomach wall, the pressure exerted on the recess 605 may cause it to inflate outward and exert a disproportionately larger force on the stomach wall (as compared to the immediate surround area, e.g., the non-recessed, flat portions 610).

In another embodiment, the size of the intragastric device 600 may be altered. For example, in a uni-intragastric device system, one intragastric device 600 may be implanted into the patient's stomach, and the single intragastric device may be sized accordingly to fit comfortably inside the patient's stomach. However, it is also possible to employ multiple, smaller devices, such as 2 or 3 objects similar to the intragastric device 600. Under this multi-intragastric device system, each intragastric device 600 may be sized such that it blocks the patient's pylorus temporarily when the intragastric device 600 comes into contact with the pylorus, and thereby slowing gastric emptying and allowing the patient to feel full for a longer period of time (and also to prevent intestinal blockage). In addition, the configuration of the intragastric device 600 may produce variations in how the intragastric device 600 sits or rotates inside the patient's stomach. Normal stomach contractions may cause the intragastric device 600 to move around or rotate about the stomach, and due to the device's configuration, different points on the inner stomach walls may be stimulated, limiting the ability of the stomach to adapt over a long period of time.

The intragastric device 600 may be constructed of rubbers, fluorosilicones, fluoroelastomers, thermoplastic elastomers or any combination thereof to improve the durability of the intragastric device 600 inside the patient's stomach. In one embodiment, the flat portions 610 and the recesses 605 may be constructed of different materials. For example, the flat portions 610 may be made of one material with one mechanical property (e.g., a rubber) while the recesses 605 may be constructed of a different material with a different mechanical property (e.g., a thermoplastic elastomer).

Alternatively, the intragastric device 600 may constructed of a continuous, thin, depressable wall of the same material, but of different thicknesses (e.g., the flat portions 610 may be thicker than the recesses 605). In one embodiment, the intragastric device 600 may be hollow inside (filled with air/inert gas) to keep the intragastric device 600 light. Alternatively, the intragastric device 600 may be filled with a liquid gel such as silicon. The materials utilized may allow for a thinner wall thickness and have a lower coefficient of friction. Thinner walls and the lower coefficient of friction allows improved natural passage of the intragastric device 600 through the gastrointestinal tract should the intragastric device 600 deflate for any reason inside the patient's stomach.

While not shown, the outer surface of the intragastric device 600 may further include additional stimulation features such as small rounded bumps or protrusions formed on the flat portions 610, quill-like extensions, and the like. These features, upon contact with the inner stomach wall of the patient may further trigger hormone release or otherwise aid the patient in feeling full.

The implantable devices described herein will be subjected to clinical testing in humans. The devices are intended to treat obesity, which is variously defined by different medical authorities. In general, the terms “overweight” and “obese” are labels for ranges of weight that are greater than what is generally considered healthy for a given height. The terms also identify ranges of weight that have been shown to increase the likelihood of certain diseases and other health problems. Applicants propose implanting the devices as described herein into a clinical survey group of obese patients in order to monitor weight loss.

For example, clinical studies on the coiled device 250 described above will be per performed with the following parameters.

Components:

Tubular sheath 254, Wire 252, End caps 256, Silicone dip coating around assembly, Adhesive

Materials:

Tubular sheath 254: Silicone rubber as defined by the Food and Drug Administration (FDA) in the Code of Federal Regulations (CFR) Title 21 §177.2600

Wire 252: Nitinol End caps 256: Delrin homopolymer Adhesive: Silicone 3166-01

Dimensions:

Tubular sheath 254: 10.16-10.32 cm (4-8 inch) overall diameter, 1.27-1.91 cm (0.5-0.75 inch) tube diameter

Purposes:

the devices are for human implant,

the devices are intended to occupy gastric space while also applying intermittent pressure to various and continually changing areas of the stomach;

the devices are intended to stimulate feelings of satiety, thereby functioning as a treatment for obesity.

General Implant Procedures:

The device is intended to be implanted transorally via endoscope into the corpus of the stomach. The device is non-fixating and requires no inflation or further manipulation once deployed. When deployed the device morphology prevents dislodgement and ensures that it remains in the gastric cavity.

However, other modes of access are contemplated, such as surgical/vascular access, various injection routes, percutaneous route, topical application, etc.

Implantation of the medical devices will occur via endoscopy.

Nasal/Respiratory administration of oxygen and isoflurane to be used during surgical procedures to maintain anesthesia as necessary.

One exemplary implant procedure is listed below.

a) Perform preliminary endoscopy on the patient to examine the GI tract and determine if there are any anatomical anomalies which may affect the procedure and/or outcome of the study.

b) Insert the introducer into the over-tube.

c) Insert the gastroscope through the introducer inlet until the flexible portion of the gastroscope is fully exited the distal end of the introducer.

d) Leading under endoscopic vision, gently navigate the gastroscope, followed by the introducer/over-tube, into the stomach.

e) Remove gastroscope and introducer while keeping the over-tube in place.

f) OPTIONAL: Place the insufflation cap on the over-tubes inlet, insert the gastroscope, and navigate back to the stomach cavity.

g) OPTIONAL: Insufflate the stomach with air/inert gas to provide greater endoscopic visual working volume.

h) Straighten the Coil, and insert fully into the over-tube.

i) Under endoscopic vision, push the Coil into the stomach.

j) Confirm deployment of the coil using visual confirmation. Ensure the tip of the coil has not entered the pylorus during delivery.

k) Insert endoscopic grasping instrumentation to adjust the Coil position in the stomach as required.

l) Perform final endoscopic inspection for any potential anomalies. Record all observations.

m) Remove the gastroscope from over-tube.

n) Remove the over-tube from the patient.

End Point Criteria:
Weight Loss
Comprehensive Metabolic Panel (CMP)
HbAlC
Lipid Panel
Tissue Samples/Response

Unless otherwise indicated, all numbers expressing quantities of ingredients, properties, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention(especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, references may have been made to patents and printed publications in this specification. Each of the above-cited references and printed publications are individually incorporated herein by reference in their entirety.

Specific embodiments disclosed herein may be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. An intragastric obesity treatment implant that stimulates the stomach walls, comprising:
an inflatable member having an inflated size that will not pass through the pyloric sphincter and a volume within the stomach of at least 400 ml and being made of a material that will resist degradation over a period of at least six months within the stomach, the inflatable member being formed as a substantially planar disc having opposed faces and a peripheral surface and a maximum width larger than the contracted width of an adult stomach so alter the shape of the stomach to conform somewhat to the inflatable member,
the implant being formed of a material which permits it to be compressed when deflated into a substantially linear delivery configuration.

2. The implant of claim 1, further including a plurality of dimples on each opposed face of the substantially planar disc defining points at which the opposed faces are connected through an inner cavity to help retain the substantially planar disc shape.

3. The implant of claim 1, wherein the opposed faces and peripheral surface are smooth.

4. The implant of claim 1, wherein the opposed faces are generally circular with a diameter, and the peripheral surface defines a thickness that is less than one-half the diameter.

5. The implant of claim 1, at least some of the opposed faces and peripheral surface are irregular in a pattern selected from the group consisting of a rounded protrusion, a quill-like extension, a recess, and combinations thereof.

6. The implant of claim 1, wherein the intragastric device is constructed out of a material selected from a group consisting of a rubber, a fluorosilicone, a fluoroelastomer, a thermoplastic elastomer, and combinations thereof.

7. An intragastric obesity treatment implant that stimulates the stomach walls, comprising:
an elongated member having a relaxed configuration that forms a coil, opposite free ends of the coil overlapping one another to permit constriction of the coil, the coil having a diameter that generally fits within the stomach of an adult patient so as to contact the interior stomach walls upon contraction thereof, the elongated member being formed of a material which permits it to be stretched into a substantially linear delivery configuration and that will resist degradation over a period of at least six months within the stomach.

8. The implant of claim 7, wherein the coil in its relaxed configuration has a diameter of between about 15-16 cm.

9. The implant of claim 7, wherein the coil comprises an inner pre-formed wire placed inside a soft and fairly flexible plastic tubular sheath.

10. The implant of claim 9, wherein the wire is Nitinol.

11. The implant of claim 9, wherein two free ends of the wire are captured by two end caps that close open ends of the sheath.

12. The implant of claim 11, wherein the assembled components of the wire, sheath and end caps are preferably coated with a flexible compound that is resistant to stomach juices.

13. The implant of claim 9, wherein the wire is embedded within the sheath with no hollow spaces therebetween.

14. An intragastric obesity treatment implant that stimulates the stomach walls, comprising:
an elongated hollow member having a relaxed configuration that forms a loop, opposite free ends of the loop being adapted to connect together, the loop having a diameter that generally fits within the stomach of an adult patient so as to contact the interior stomach walls upon contraction thereof, the elongated hollow member being formed of a material which permits it to be stretched into a substantially linear delivery configuration and that will resist degradation over a period of at least six months within the stomach.

15. The implant of claim 14, wherein the elongated hollow member further including perforations along its length to permit ingress of stomach fluids.

16. The implant of claim 14, wherein the elongated hollow member has a distal end connector with a lumen, and a proximal end connector with a lumen and a side aperture spaced from the proximal end connector, the implant further including a tether that extends through the hollow lumen on the distal end connector and is secured therein, and passes in through the proximal end connector lumen and outward through the side aperture, the proximal and distal end connectors being brought together upon pulling the tether taut.

17. The implant of claim 14, wherein the loop has a diameter of between about 15-16 cm.

18. The implant of claim 14, further including a spring placed inside the elongated hollow member to provide resiliency and prevent kinking of the elongated hollow member.

19. The implant of claim 14, wherein the loop is constructed by cutting a plastic tube in two to form two smaller diameter half tubes that fit inside a larger diameter whole tube.
